(19) United States
(12) Patent Application Publication
(10) Pub. No.: US 2013/0225630 A1
(43) Pub. Date: Aug. 29, 2013

(54) TOPICAL RAPAMYCIN FOR TREATMENT OF FACIAL ANGIOFIBROMAS IN TUBEROUS SCLEROSIS

(71) Applicants: Joyce M.C. Teng, Madison, WI (US); Anna K. Haemel, Madison, WI (US); Marla J. Ahlgrimm, Fitchburg, WI (US)

(72) Inventors: Joyce M.C. Teng, Madison, WI (US); Anna K. Haemel, Madison, WI (US); Marla J. Ahlgrimm, Fitchburg, WI (US)

(21) Appl. No.: 13/852,773

(22) Filed: Mar. 28, 2013

Related U.S. Application Data
(63) Continuation of application No. 13/168,634, filed on Jun. 24, 2011.

(51) Int. Cl. A61K 31/439

(52) U.S. Cl. CPC A61K 31/439
USPC 514/291

(57) ABSTRACT

The present disclosure provides for a method and a topical composition to treat facial angiofibromas in Tuberous Sclerosis by applying from about 0.25% to about 2% rapamycin to a small body surface area.

### CROSS-REFERENCE TO RELATED APPLICATIONS

This is a continuing application that claims benefit to U.S. application Ser. No. 13/168,634, filed on Jun. 24, 2011, which is a non-provisional application that claims benefit to U.S. Provisional Application No. 61/358,205 filed on Jun. 24, 2010. Both of these applications are hereby incorporated by reference.

### FIELD

The present invention provides a method and a topical composition by applying topical rapamycin to treat facial angiofibromas, a cutaneous manifestation of Tuberous Sclerosis (TS) that can be both debilitating and disfiguring, and have historically been resistant to medical and surgical treatments.

### BACKGROUND

Facial angiofibromas, which occur in 70% to 80% of patients with TS, appear as innumerable pink papules that progressively enlarge and multiply over time. The lesions, which are highly visible markers of disease, may spontaneously bleed, impair vision, and cause emotional distress. Current treatment options for facial angiofibromas include destructive approaches such as dermabrasion, surgical excision, and laser therapy. Current therapies are not effective in preventing early lesions. Many TS patients have numerous large angiofibromas that tended to recur despite destructive approaches, and develop many new lesions at a rapid rate. Furthermore, it is necessary to balance aggressive therapy against the risk of significant permanent scarring. Also, sedation poses a high risk because of many TS patients' underlying seizure disorder. Some previous surgical procedures required antiepileptic loading and prolonged intubation. In light of all the risks and possible complications, TS patients' recalcitrant tumors present a significant therapeutic challenge.

### BRIEF DESCRIPTION OF THE DRAWINGS

The patent or application file contains at least one photograph executed in color. Copies of this patent application publication with color photographs will be provided by the Office upon request and payment of the necessary fee.

FIGS. 1A and 1B depict an examination at baseline before topical rapamycin therapy; and FIGS. 1C and 1D depict an examination after 12 weeks of topical rapamycin therapy showing reduced angiofibromas in number and size.

### SUMMARY OF THE INVENTION

Briefly, therefore one aspect of the present disclosure provides a method of treating facial angiofibromas in Tuberous Sclerosis. The method comprises the step of applying an effective amount of topical rapamycin by topical administration to skin area to be treated on a subject. The said topical rapamycin is a composition comprising from about 0.25% to about 2% by weight of rapamycin. Generally, the topical rapamycin composition may further comprise a dermatologically acceptable carrier. The method provided is applicable to mammal, and in some embodiment, the mammal is a human.

Another aspect of the present disclosure provides a method of reducing cutaneous vascular lesion. The method comprises applying an effective amount of topical rapamycin by topical administration to skin area to be treated on a subject. The said topical rapamycin is a composition comprising from about 0.25% to about 2% by weight of rapamycin. Generally, the topical rapamycin composition may further comprise a dermatologically acceptable carrier. The method provided is applicable to mammal, and in some embodiment, the mammal is a human.

Yet another aspect of the present disclosure provides a topical composition for reducing cutaneous vascular lesion, said composition comprises an effective amount of rapamycin; and a dermatologically acceptable carrier. The effective amount of rapamycin in said composition is from about 0.25% to about 2% by weight of rapamycin. Generally, the dermatologically acceptable carrier in said composition is selected from the group consisting of solvent, lubricant, emollient, emulsifier, moisturizer, thickening wax, softener, fragrance, preservative, and artificial color(s). In one embodiment, the dermatologically acceptable carrier of said composition is pertrolatum. In addition, the topical composition provided herein can treat including facial angiofibroms, infantile hemangioma and Kaposi sarcoma, all of which are cutaneous vascular lesions. The provided topical composition has the effective ingredient decreasing the formation of blood vessels in angiogenesis.

### DETAILED DESCRIPTION

The present disclosure provides a method to treat facial angiofibromas in Tuberous sclerosis (TS) by applying topical rapamycin.

Rapamycin, with a tradename known as Sirolimus, is a macrolide antibiotic isolated from Streptomyces hygroscopicus that has demonstrated immunosuppressive activity. Rapamycin has been traditionally used in transplant recipients. Rapamycin belongs to a novel group of molecules known as the mTOR (mammalian target of rapamycin) inhibitors and is approved for use in renal transplantation and drug-eluting stents in the United States. The molecular mechanisms of rapamycin are complex, and its signaling pathways have only recently been partially understood. In addition to its recognized immunosuppressive effects, this molecule demonstrates antineoplastic activity both in vitro and in vivo. Rapamycin exerts this effect by decreasing production of the proangiogenic molecule VEGF (vascular endothelial growth factor), which is implicated in many cancers, as well as by inhibiting its downstream signaling. Other topical immunosuppressive agents such as calcineurin do not demonstrate these effects.

Furthermore, rapamycin appears to correct aberrant signaling in a variety of pathways that regulate cell growth and apoptosis, including those activated in some tumor states, such as TS.5. Tuberous sclerosis is an autosomal dominant tumor syndrome that results from mutations in the tumor suppressors hamatin (TSC1) or tuberin (TSC2). Hamartin and tuberin normally suppress mTOR, which increases cell cycle progression when it is released from negative regulation. The loss of tumor suppressive function in TS leads to the formation of multiple tumors of the internal organs and skin.

The present disclosure provides for a treatment method, that is, applying topical rapamycin ointment for recalcitrant facial angiofibromas in patients with TS. The treatment appears to be well tolerated with no evident local or systemic adverse effects. Furthermore, it avoids the risks of general anesthesia and surgical complications and appears to produce more sustained effects than procedural treatments.

Accordingly, one aspect of the present disclosure is the use of compound as being capable to treat cutaneous vascular lesions, which include facial angiofibromas in patients with TS.

Also, one aspect of the present disclosure is a method for treating recalcitrant facial angiofibromas in TS with regimen applying topically to an affected local body surface area with formulation that comprises topical rapamycin at a range from about 0.1% to about 2%. In another embodiment the topical rapamycin at a range from about 0.25% to about 2%.

Another aspect of the present invention provides a topical compound for treating cutaneous vascular lesions, which include facial angiofibromas in patients with TS. The provided compound contains an active ingredient rapamycin mixed into a dermatologically acceptable lotion or cream base carrier for topical application and to deliver the active ingredients to skin being treated. Exemplary carrier formulations include, not limited to, solvent, such as water; lubricant and emollient, such as mineral oil; emulsifier and moisturizer, such as Sorbitan sesquioleate and petrolatum; thickening wax, such as ceresin; softener, such as lanolin; fragrance; preservative, such as methylparaben and propylparaben; and artificial colors.

Examples include but are not limited to application of topical rapamycin for facial angiofibromas in TS. Rapamycin decreases the formation of blood vessels in tumor cell (angiogenesis). In addition, rapamycin inhibits the translation of key mRNAs of proteins required for cell cycle progression from G₁ to S phase, and thus prevents cell division by leading to growth arrest at the G₁ phase of the cell cycle. Given the effects of rapamycin therapy on both angiogenesis and cell division, topical preparations of rapamycin therefore have broader application for a variety of benign (eg, infantile hemangioma) and malignant cutaneous vascular lesions, such as infantile hemangioma, Kaposi sarcoma. Infantile hemangioma is the most common tumor of orbit and periorbital areas in childhood. Kaposi's sarcoma (KS) is a tumor caused by Human herpesvirus 8 (HHV8), also known as Kaposi's sarcoma-associated herpesvirus (KSHV). Kaposi's sarcoma (KS) is a systemic disease which can present with cutaneous lesions with or without internal involvement. In addition, Capillary Malformations (CMs) (or commonly known as port-wine stain) is also a vascular anomaly treatable by rapamycin. Capillary Malformations are present at birth and come in a variety of sizes and locations, and do not undergo spontaneous resolution. Capillary malformations typically grow in proportion to the growth of the child. Capillary Malformation stains may involve any area of the body and are usually pink or reddish during infancy, but often darken with advancing age.

### Example 1

A 16-year-old girl presented with a complex medical history that was related to both systemic and cutaneous manifestations of TS. In addition to her cutaneous manifestations of progressive facial angiofibromas that she had had since she was 5 years old and, more recently, gingival fibromas, she also had renal angiolipomas, severe mental retardation, and epilepsy with complex partial seizures that generalize. She had been placed on a regimen of several antiepileptic medications, currently oxcarbazepine and divalproex sodium, for control of intractable seizures. At 10 months of age, she underwent open heart surgery for removal of a rhabdomyoma that was blocking her pulmonary artery. At 13 years of age, she underwent endoscopic removal of a cranial intraventrical mass, with pathologic examination demonstrating subependymal giant cell astrocytoma, followed by placement of a ventriculostomy for relief of obstructive hydrocephalus. Also, she underwent multiple shave excisions and repeated treatments with both pulsed dye and carbon dioxide lasers, with at least 1 treatment per year over the last 3 years, to control bleeding and rapid progression of facial angiofibromas. Despite these aggressive interventions, her facial lesions remained prominent, progressive, and disfiguring, with a tendency toward recurrence and new lesions. The procedural treatments had no lasting effects on the progression of her condition.

A range of topical rapamycin ointment with concentrations from about 0.25% to 2% is compounded for compassionate use in the affected patient. Rapamycin was extracted from tablets to be used for the ointment, and petrolatum was used as the major ingredient in the vehicle to minimize irritation. The compounded medication is intended for use within 3 months. Approximately 0.5 g of ointment was used with each application, for a total of 5 mg of topical rapamycin.

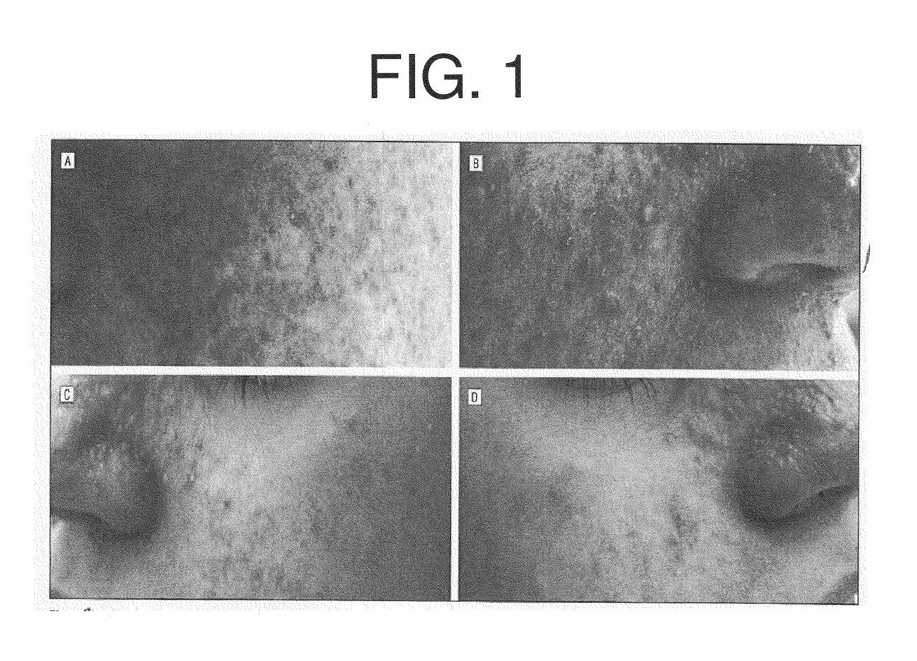

Before beginning the topical treatment, the patient was noted to have numerous facial angiofibromas measuring approximately 0.5 to 4 mm in greatest dimension and diffusely involving the glabella and temples, with more prominent involvement of the central area of the face and the nasal bridge (FIG. 1A and B). Topical rapamycin therapy with 1% ointment was initiated twice daily. At 1 week, decreased erythema in the patient was reported. Shortly thereafter, the patient's skin texture presented gradual improvement.

At the 6-week follow-up visit, the patient was noted to have a reduced number of angiofibromas and improved facial erythema. The vascular papules on both cheeks were much smaller. The lesions on the temples and forehead were completely resolved. This striking improvement had never been achieved with previous procedural treatments. At the 12-week reassessment, the patient showed a sustained effect, with continued improvement in skin texture (Figure, C and D). Laboratory tests were performed at 6 weeks and 12 weeks to evaluate potential systemic adverse effects. Complete blood cell counts and the results of a complete metabolic panel remained stable at baseline. The serum rapamycin level remained under 2 ng/ml, (below the limits of detection; reference range, 4-20 ng/ml.). Therefore, no measurable systemic absorption was detected after 3 months' application of 1% topical rapamycin to approximately 1% of the body surface area (BSA). Therefore, topical rapamycin therapy could be an effective treatment for facial angiofibromas, with minimal systemic toxic effects.

A reverse-phase high-performance liquid chromatography (HPLC) method was used for therapeutic drug monitoring of rapamycin. The monitoring method was described in Therapeutic Monitoring of Sirolimus in Human Whole-Blood Samples by High-Performance Liquid Chromatography (Saber Maleki, et al., 2000). This reference is hereby incorporated by reference in its entirety.

The patient did not demonstrate detectable serum rapamycin levels after 3 months of application to her face. Furthermore, there were no changes in blood cell counts or chemistry profiles from baseline or other evidence of systemic adverse effects of rapamycin therapy. Therefore, it is unlikely that the 1% preparation applied to a limited BSA would result in systemic toxic effects.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of a preferred embodiment and best mode of the invention known to the applicants at this time of filing the application has been presented and is intended for the purposes of illustration and description. It is not intended to be exhaustive or limit the invention to the precise form disclosed and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out this invention, but that the invention will include all embodiments falling within the scope of the appended claims.

What is claimed is:

1. A method of treating facial angiofibromas in Tuberous Sclerosis comprising:
applying an effective amount of topical rapamycin by topical
administration to skin area to be treated on a subject so that the
topical rapamycin is not systemically absorbed by the subject.

2. The method of claim 1, wherein the topical rapamycin is a composition comprising from about 0.1% to about 2% by weight of rapamycin.

3. The method of claim 1, wherein the topical rapamycin is a composition comprising from about 0.25% to about 2% by weight of rapamycin

4. The method of claim 1, wherein the topical rapamycin is a composition further comprising a dermatologically acceptable carrier.

5. The method of claim 1, wherein the subject is a mammal.

6. The method of claim 5, wherein the mammal is a human.
